(19) United States
(12) Patent Application Publication
(10) Pub. No.: US 2019/0038447 A1
(43) Pub. Date: Feb. 7, 2019

(54) SYSTEMS, METHODS AND DEVICES FOR SUPPORTING LIMBS OF A WEARER

(71) Applicant: Shock Doctor, Inc., Minnetonka, MN (US)

(72) Inventor: James J. Landi, West Orange, NJ (US)

(21) Appl. No.: 15/759,049

(22) PCT Filed: Sep. 9, 2016
(86) PCT No.: PCT/US16/51077

§ 371 Date: Mar. 9, 2018

Related U.S. Application Data
(60) Provisional application No. 62/216,879, filed on Sep. 10, 2015.

(51) Int. Cl. A61F 5/01

(52) U.S. Cl. CPC A61F 5/0109

(57) ABSTRACT

A support assembly, comprising a sleeve configured to surround a portion of a limb of a wearer; the sleeve having a main body defining a first opening, a second opening, an inner surface, and an outer surface, wherein the first and second openings are configured to receive a limb of a wearer. The support assembly includes a support insert configured to engage the sleeve and be held in position against a limb of a wearer by the sleeve; and a strap configured to be positioned around the support insert and sleeve and retain the support insert against the portion of a limb of a wearer.

### CROSS REFERENCE TO RELATED APPLICATION

This PCT application claims priority to U.S. Patent Application Ser. No. 62/216,879 filed on Sep. 10, 2015, which is incorporated herein by reference in its entirety for all purposes.

### BACKGROUND

Over time, various externally applied devices for modifying the structural or functional characteristics of a wearer's neuromuscular or skeletal system have been developed, including knee, ankle, and shoulder braces or sleeves, among others. In certain instances they may assist a wearer's movement, reduce weight bearing forces, or provide easier movement capability to a wearer.

For example, an athletic sleeve may be worn around a wearer's limb to provide compression to the limb. An athletic sleeve may also be worn around a wearer's joint such as a knee, wrist, ankle, or elbow to provide lateral or torsional support to the joint and/or to hold the joint in alignment. In some cases, an athletic sleeve may hold snugly to a joint such as a knee or elbow and improve performance while the wearer is engaged in physical activity. However, in some cases, an athletic sleeve may not provide adequate support to a wearer who has suffered an injury or may not provide targeted support to a wearer whose joint may require support in a specific location.

Additionally or alternatively, a support brace may be worn to provide greater weight bearing support to a limb or joint of a wearer than an athletic sleeve alone. In some cases, a support brace may be made of rigid material that provides greater structural support than an athletic sleeve, or may be designed to keep a joint in alignment during physical activity. However, certain support braces currently available may not provide suitable or adequate support to a wearer who may require relief in a specific part of a limb or joint.

### SUMMARY

Disclosed herein is a support assembly, comprising a sleeve configured to surround a portion of a limb of a wearer; the sleeve having a main body defining a first opening, a second opening, an inner surface, and an outer surface, wherein the first and second openings are configured to receive a limb of a wearer. The support assembly includes a support insert configured to engage the sleeve and be held in position against a limb of a wearer by the sleeve; and a strap configured to be positioned around the support insert and sleeve and retain the support insert against the portion of a limb of a wearer.

Also disclosed herein is an athletic brace, comprising a sleeve configured to surround at least a portion of a wearer's limb, and configured to provide stabilizing support to the portion of a wearer's limb. The athletic brace includes a support pad molded to conform to an outer shape of the portion of a wearer's limb, the support pad configured to be held in place along the wearer's limb by the sleeve; and a strap assembly configured to attach to the sleeve and reinforce the support pad against the wearer's limb.

Also disclosed herein is a support system for a limb of a wearer, comprising a wrap having a first and second opening and an inward facing surface and an outward facing surface. The support system includes a cushioned insert and a strap system configured to engage the outward facing surface of the resilient wrap. The support system is constructed such that the wrap is configured to surround a limb of a wearer and provide a compressive force to the limb of a wearer, the cushioned insert is held against the limb of a wearer by the compressive force provided by the wrap, and the strap system maintains the cushioned insert in position against the limb of a wearer.

While multiple embodiments are disclosed, still other embodiments of the present invention will become apparent to those skilled in the art from the instant disclosure, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a perspective view of a support assembly, according to some embodiments.

FIG. 2 is a perspective view of a sleeve of the support assembly of FIG. 1, according to some embodiments.

FIG. 3 is a perspective view of an insert of the support assembly of FIG. 1, according to some embodiments.

FIG. 4 is a rear view of an insert of the support assembly of FIG. 1, according to some embodiments.

FIG. 5 is a front view of an insert of the support assembly of FIG. 1, according to some embodiments.

FIG. 5A is a diagram illustrating a cross-section view taken along the plane shown by the line A-A in FIG. 5, according to some embodiments.

FIG. 5B is a diagram illustrating a cross-section view taken along the plane shown by the line B-B in FIG. 5, according to some embodiments.

FIG. 6 is a perspective view of a strap of the support assembly of FIG. 1, according to some embodiments.

FIG. 7 is a perspective view of components of a support assembly, according to some embodiments.

FIG. 8 is a perspective view of a sleeve of the support assembly of FIG. 7, according to some embodiments.

FIG. 9 is a perspective view of an insert of the support assembly of FIG. 7, according to some embodiments.

FIG. 10 is a rear view of an insert of the support assembly of FIG. 7, according to some embodiments.

FIG. 11 is a front view of an insert of the support assembly of FIG. 7, according to some embodiments.

FIG. 11A is a diagram illustrating a cross-section view taken along plane shown by the line A-A in FIG. 11, according to some embodiments.

FIG. 11B is a diagram illustrating a cross-section view taken along the plane shown by the line B-B in FIG. 11, according to some embodiments.

FIG. 12 is a perspective view of a strap of the support assembly of FIG. 7, according to some embodiments.

FIG. 13 is a front view of a support assembly, according to some embodiments.

FIG. 14 is a side view of a support assembly, according to some embodiments.

FIG. 15 is a side view of a support assembly, according to some embodiments.

### DETAILED DESCRIPTION

Various aspects of the present disclosure relate to a support assembly that may be worn to protect and/or support a wearer's limb, including a joint, for example. The support assembly optionally applies tension and/or compression to the limb and/or the joint. In some embodiments, the support assembly is molded to fit and support the elbow, knee, or any joint that has suffered an injury. In some embodiments, the support assembly provides a support system for a wearer who has suffered a meniscus tear, or has an unstable ligament, or an injury affecting lateral stability.

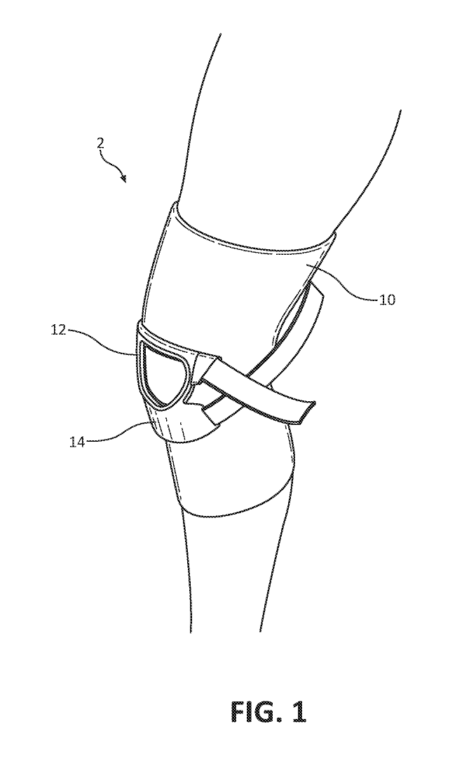

FIG. 1 is a perspective view of a support assembly 2. As shown in FIG. 1, in some embodiments, a support assembly 2 includes a sleeve 10, an insert 12 and a strap 14. In general terms, the sleeve 10 is configured to maintain the insert 12, and the strap 14 assists with maintaining a position of the insert 12 and provides additional support as desired. The insert 12 is optionally molded and designed to help support various joint injuries and can be provided with a unique design and shape for a variety of injuries. The strap 14 holds the insert 12 against a wearer's joint and provides adjustable tension to retain the insert 12 in a desired position.

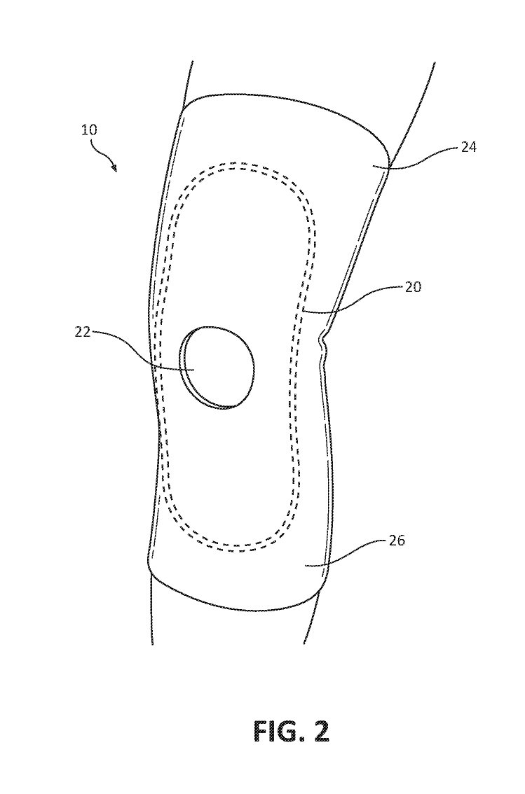

FIG. 2 is a perspective view of a sleeve 10, according to some embodiments. As shown, the sleeve 10 has a tubular body 20 defining a central opening 22 corresponding to the patella, or knee cap of a wearer. The sleeve 10 is optionally formed of elastic material in a “one-size-fits-all” or “one-size-fits-most” configuration, sometimes termed “a universal sleeve.” The sleeve 10 is worn around a wearer's limb and, according to some embodiments, is tubular in shape with a first end defining a first opening 24 and a second end defining a second opening 26. The first and second openings 24, 26, of the sleeve 10 are optionally the same, or different shapes (e.g., with the first opening 24 being larger than the second opening 26 as shown in FIG. 2).

In some embodiments, the sleeve 10 may be worn by inserting a wearer's limb, such as an arm or leg, through either the first opening 24 or second opening 26 and advancing the sleeve 10 along the outside of the wearer's limb until the sleeve 10 reaches the desired location along the limb or joint. In some embodiments, the sleeve 10 is made of material that will allow the sleeve 10 to remain snug around a wearer's limb and provide support to a limb or joint. For example, a sleeve 10 may be made of an elastic material, such as neoprene, that provides compression to a wearer's limb or joint once in place.

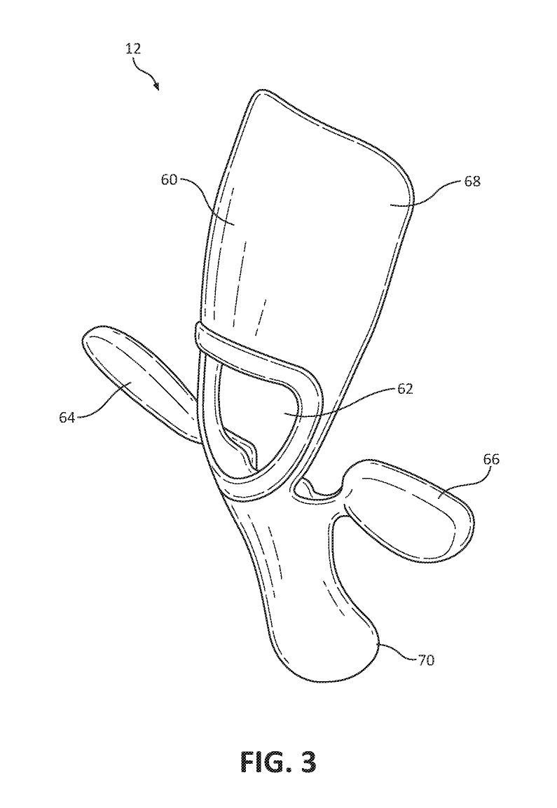

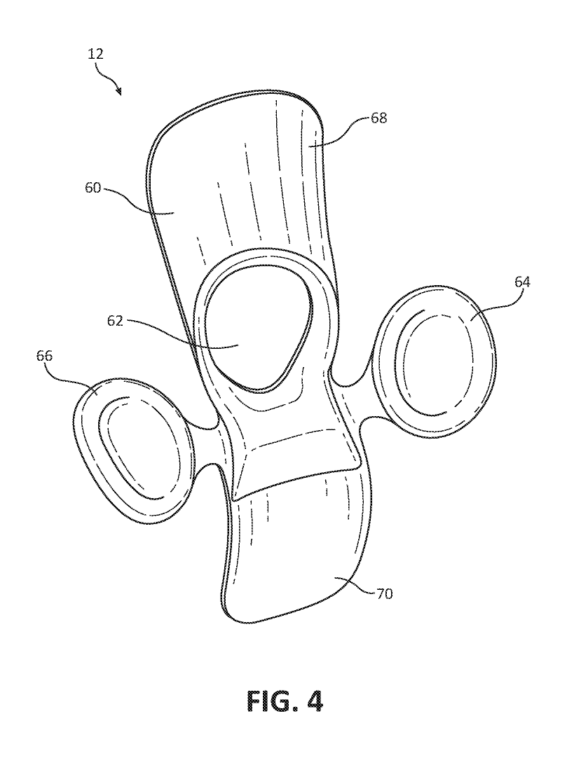

For illustrative purposes, FIGS. 3, 4, and 5 contain an example insert 12 that is used with some embodiments of the support assembly 2 shown in FIG. 1. In some embodiments, an insert 12 is constructed to conform to the shape of the outer surface of a wearer's limb or joint, and provides support and/or pressure along certain areas of the limb or joint. In some embodiments, the insert 12 is constructed to support part of a limb or joint from one or multiple sides. In some embodiments, the insert 12 completely encompasses a wearer's limb or joint.

In some embodiments, the insert 12 is constructed to cover the medial and lateral sides of a wearer's knee. In some embodiments, the insert 12 is constructed to be positioned on the medial and lateral sides of a wearer's knee beside the knee joint and the kneecap. In some embodiments, the insert 12 is constructed to be positioned superior and inferior to the wearer's knee or knee cap. In some embodiments, the insert 12 is constructed to be positioned on the anterior side of the leg, superior and inferior the knee joint and the knee cap.

As shown in FIGS. 3, 4, and 5, the insert 12 is shaped to be positioned against various parts of a wearer's limb and joint. The insert 12 includes a main body 60 that provides support to a wearer's leg and knee. The main body 60 optionally defines an opening 62 that corresponds to the knee cap or patella of a wearer. In some embodiments, an opening 62 corresponds to the wearer's elbow, wrist, or ankle.

The insert 12 is constructed from any suitable material that provides support to a user's anatomy, yet remains comfortable when worn. Insert material is also chosen from material that will flex or move with the user's movements. Some example materials that the insert 12 is made from include foam, rubber, or a polymeric material.

In some embodiments, the insert 12 includes lateral, medial, superior and inferior support elements 64, 66, 68, 70 attached to the main body 60. The support elements 64, 66, 68, 70 are constructed to conform, for example, to the lateral, medial, superior, and inferior sides of a wearer's knee respectively. The support elements 64, 66, 68, 70 are suitably shaped and positioned to support the knee cap and knee joint.

In some embodiments, the insert 12 structure provides pressure to certain points along a limb or joint. For example, the support elements 64, 66, 68, 70 of the insert 12 are constructed to apply pressure from the lateral, medial, superior, or inferior sides of the knee joint respectively, to increase support for the joint. In some embodiments, the insert 12 may be constructed to prevent certain parts of the joint, such as the kneecap, from being pressed against the joint during physical activity. For example, the opening 62 allows parts of a wearer's joint to be positioned through the opening 62 for additional fit, and have support elements 64, 66, 68, 70 positioned around or cradle suitable parts of a wearer's joint. As shown in FIGS. 3, 4, and 5 support elements 64, 66 that correspond to the lateral and medial sides of the wearer's knee are positioned in the plane of the main body 60. However, when worn, support elements 64, 66 are turned inward, so they are positioned perpendicular to the plane of the main body 60, and lay flat against the medial and lateral sides of the knee.

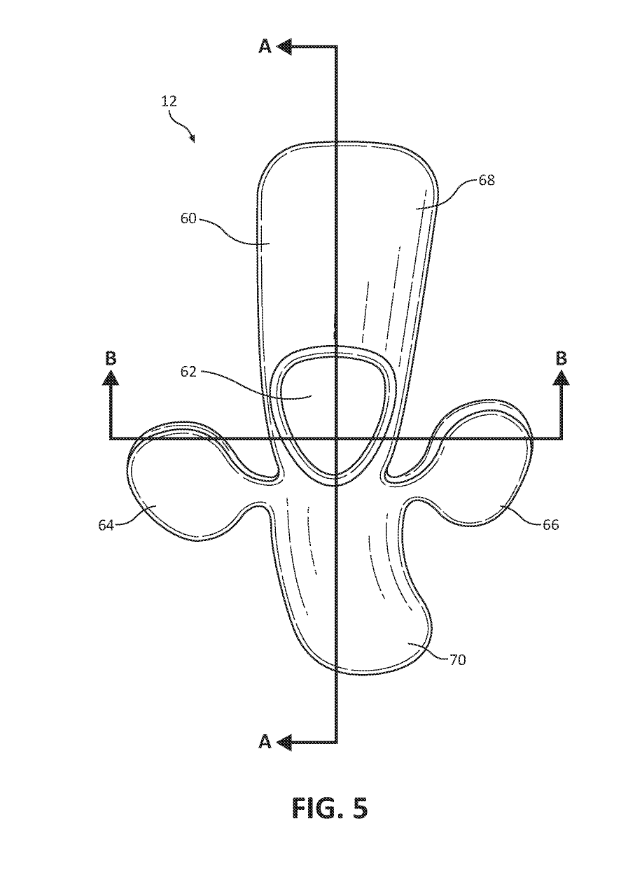

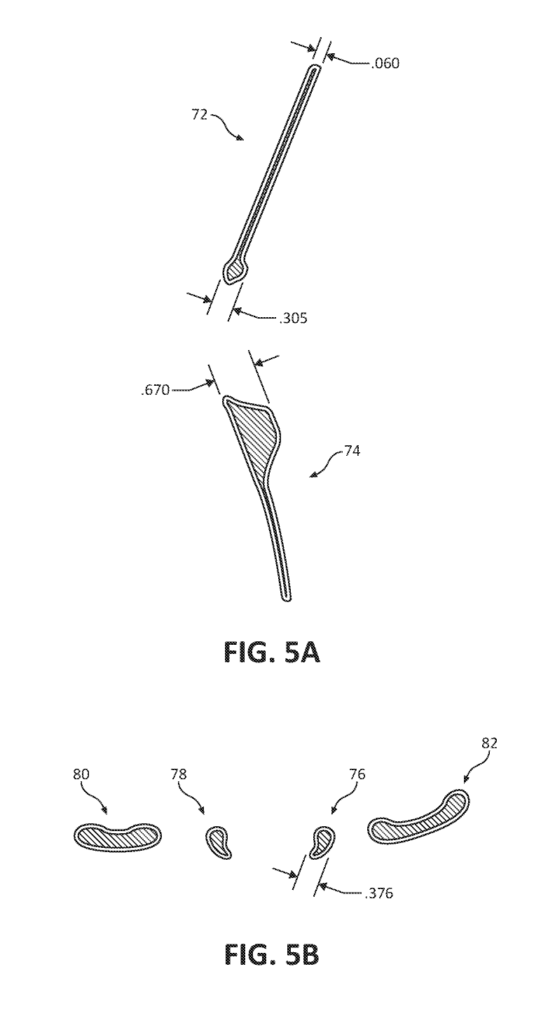

FIG. 5A is a cross-sectional view of the insert 12 in the plane of the lines marked by arrows A-A in FIG. 5. As shown in FIG. 5A, the thickness of the insert 12 varies in specific locations to provide pressure to certain points along a wearer's knee and knee cap. For example, the portion of the insert 12 corresponding to the area superior 72 to the wearer's knee corresponds to the profile of the space superior to the wearer's knee cap and leg. Additionally, the portion of the insert 12 corresponding to the area inferior 74 to the wearer's knee corresponds to the profile of the space inferior to the wearer's knee cap and between the knee cap and leg.

FIG. 5B is a cross-sectional view of the insert 12 in the plane of the lines marked by the arrows B-B in FIG. 5. As shown in FIG. 5B, the thickness of the insert 12 varies in specific locations to provide support or pressure to certain points along a wearer's knee cap and knee joint. For example, a first portion 76 of the insert 12 may be configured to correspond to the profile of the lateral side of the wearer's knee cap and leg. Additionally, a second portion of the insert 12 may be configured to correspond to the profile of the space medial to the wearer's knee cap and leg. Support elements 64, 66 (shown in FIG. 5) also have a thickness that provides support when the insert 12 is positioned against the sides of a wearer's knee. As shown in FIG. 5B, a cross-sectional view of the insert of FIG. 5 illustrates the shaped thickness of support elements 64, 66 (shown in FIG. 5) that allow them to support a wearer's knee from the medial 80 and lateral 82 sides of the wearer's knee.

In some embodiments, the insert 12 is constructed to conform to the outer shape of a wearer's elbow, ankle, or wrist. In an example, the insert 12 is constructed to fit around the wrist of a wearer and provide support to the wrist bones. In another example, the insert 12 may be constructed to fit in the palm, around the thumb, or any other area of a wearer's hand. In yet another example, the insert 12 may be constructed to support a wearer's ankle.

In some embodiments, the insert 12 is a pad that has been custom molded to fit a particular wearer's joint. For example, a potential wearer's anatomy, such as a leg, arm, knee, elbow, or torso is first scanned to determine the outer shape of the body part. Using data from the scan, a three dimensional model is created that corresponds to the outer surface of the scanned body part. The corresponding model is then used to design a custom molded insert 12 for the particular shape of a particular user's joint.

The three dimensional model is also used to determine what shape an insert 12 must be in order to provide custom support to a particular area of a wearer's body part. For example, if a potential wearer has suffered an injury that requires greater support or pressure at a particular area, a scan of the wearer's anatomy may provide the necessary location of pressure points. The three dimensional model is used to design and mold an insert 12 that corresponds to provide additional support to these pressure points. Examples of some methods of forming pads suitable for use as inserts are provided in U.S. Patent Application No. 2011/0056004, the entire contents of which are incorporated herein by reference.

In some embodiments, the insert 12 is held in place by joining the insert 12 to the sleeve 10. For example, the sleeve 10 has pockets or compartments for securely holding an insert 12. In some embodiments, the sleeve 10 has compartments on the inside of the sleeve 10 such that a user places an insert 12 inside these compartments. In some embodiments, the insert 12 is attached to the sleeve 10 by a fastener such as a clasp or clip. In some embodiments, the sleeve 10 is constructed with straps or belts for removeably holding an insert 12 in place. The pockets or compartments are constructed to hold the insert 12 in a particular positon against the wearer's limb or joint. The sleeve 10 is optionally constructed to hold the insert 12 in a fixed position relative to the sleeve 10. In some embodiments, the sleeve 10 is constructed to allow the insert 12 to be repositioned with respect to the sleeve. The sleeve 10 is constructed to hold a variety of inserts 12, such that the same sized sleeve 10 may be used by a number of wearers.

In some embodiments, the sleeve 10 is constructed with compartments configured to hold an insert 12 in position against the wearer's limb or joint while the wearer is engaged in physical activity. For example, the compartments are made from elastic or flexible material that allows the tubular body 20 of the sleeve 10 to move with the wearer's body yet maintains the insert 12 in place to provide support for the wearer's joint.

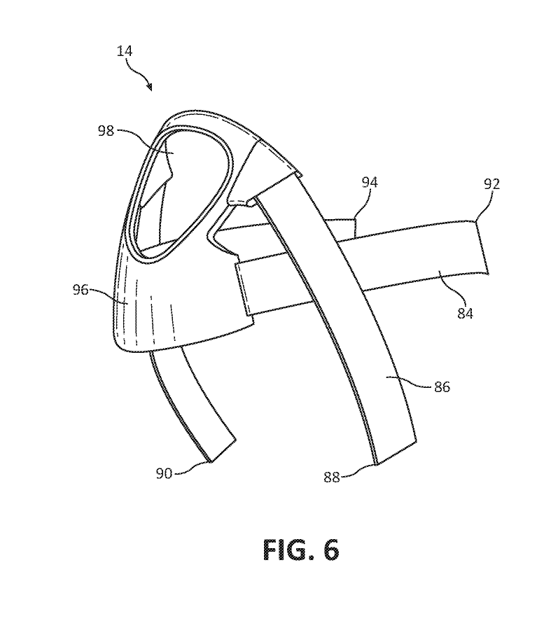

In some embodiments, a strap 14 is used in conjunction with the sleeve 10 to hold the insert 12 in position. FIG. 6 shows a perspective view of the strap 14. The strap 14 is used in conjunction with the sleeve 10 to apply adjustable tension, fit, and support to the wearer's limb or joint. The strap 14 is also used to hold the insert 12 in place against the wearer's limb or joint. In some embodiments, the strap 14 is constructed to apply adjustable tension such that the insert 12 is held snug against a wearer's limb.

In some embodiments, the strap 14 is constructed as a separate component from the sleeve 10. In some embodiments, the sleeve 10 is constructed with the strap 14 attached to the sleeve 10. In some embodiments, the strap 14 is attached to the sleeve 10 along the entire length of the strap 14. In some embodiments, the strap 14 is attached to the sleeve 10 only in certain locations. For example, the strap 14 may have portions that are not attached to the sleeve 10, thus allowing the strap 14 to move independent of the sleeve 10 at the unattached locations. The strap 14 may attach to the sleeve 10 in one or more places and applies tension between the attached locations.

The strap 14 is attached to the sleeve 10 by any suitable attachment method such as sewing, or using clips, clasps, hook and loop fasteners sold under the trade name “VELCRO,” or buttons, for example. In some embodiments, the strap 14 is configured to allow a wearer to partially or completely remove the strap 14 from the sleeve 10, for example to change the position where the strap 14 connects to the sleeve 10.

In some embodiments, the strap 14 is a band positioned around the insert 12 and the sleeve 10. The band thus applies compression to the insert 12 in a direction normal to the axial direction of a wearer's limb and holds the insert 12 against the wearer's limb. In some embodiments, the strap 14 is constructed such that it wraps completely around the sleeve 10 and a wearer's limb or joint. Alternatively, the strap 14 can be constructed to wrap partly around the wearer's limb or joint.

As illustrated in FIG. 6, in some embodiments, the strap 14 includes multiple bands. For example, the strap 14 may be constructed with a first band 84 that travels under or inferior to a wearer's knee cap, and a second band 86 that travels over or superior to a wearer's knee cap. When using multiple bands to form a strap 14, a variety of optional configurations are possible for positioning the bands in relation to the joint and insert 12. For example, first and second bands 84, 86 may cross over each other forming an X in the front of a knee cap, or can cross on the sides of the knee. In another example, first and second bands 84, 86 may be constructed to each have first ends 88, 92 attach at the lateral or posterior side of a wearer's knee, traverse the anterior portion of the knee and each have second ends 90, 94 attach to the posterior or medial side of the knee.

In a strap 14 with first and second bands 84, 86, the first and second bands 84, 86 work in conjunction to keep the insert 12 in a suitable position. The strap 14 holds the insert 12 in place by preventing movement in a first axial direction along the length of the wearer's limb with the first band 84, and prevents movement in a second axial direction along the length of the wearer's limb with the second band 86.

In some embodiments, the strap 14 is configured to correspond to the outer shape of the insert 12 and provide a complimentary fit to the insert 12. The strap 14 is optionally configured to be positioned within shaped depressions in the outer surface of the insert 12 to further assist the strap 14 in preventing lateral or torsional movement of the insert 12 in relation to the wearer's joint.

In some embodiments, a retaining portion 96 is constructed between the first band 84 and second band 86 and connects the first band 84 and the second band 86. The retaining portion 96 is configured to correspond to the outer shape of the insert 12 such that the strap 14 prevents lateral or torsional movement of the insert 12 in relation to the wearer's limb. In some embodiments, the retaining portion 96 defines an opening 98 to allow parts of a wearer's joint to be received inside the opening 98. The opening 98 is optionally shaped to support a kneecap or patella, or an ankle, elbow, or wrist. The opening 98 is configured to correspond to the outer shape of the insert 12 and retain it in position against the wearer's limb or joint.

In some embodiments, the strap 14 is configured to allow a wearer to adjust the tension applied by the strap 14 to the wearer's limb or joint. For example, the strap 14 allows a wearer to adjust the length of the strap 14 using a clasp or clip. Alternatively, the strap 14 is repositionable with hook and loop fasteners, to allow a wearer to reposition where the ends 88, 90, 92, 94 of the strap 14 attach to the sleeve 10. Using repositionable attachments allows a wearer to reposition the strap 14 at various locations on the sleeve 10 and thus allows the wearer to adjust the position of the strap 14 in relation to the wearer's limb. The wearer can also adjust the angle of the strap 14 in relation to the wearer's limb or joint and adjust the strap tension and the pressure points on the insert 12.

As previously shown in FIG. 1, when worn, the support assembly 2 includes the sleeve 10, insert 12, and strap 14 coupled together. The sleeve 10, insert 12, and strap 14, in conjunction, provide tailored support for a wearer. A suitable insert 12 is chosen for a wearer's particular needs. The insert 12 may be a foam body molded or designed to help support a joint injury with a unique design for each particular wearer. The insert 12 is produced to be used with a variety of sleeves 10 and may be provided separately from the support assembly. The insert 12 is placed into the sleeve 10 by the wearer and held in place using connectors, pockets, clasps, or clips. The sleeve 10 containing the insert 12 is placed onto a wearer's limb and positioned around a wearer's joint. The strap 14 is positioned around the sleeve 10 and insert 12 and connected to the sleeve 10 to supply adjustable tension, fit, and support to the sleeve 10 and insert 12, and holds the insert 12 in place against the wearer's joint.

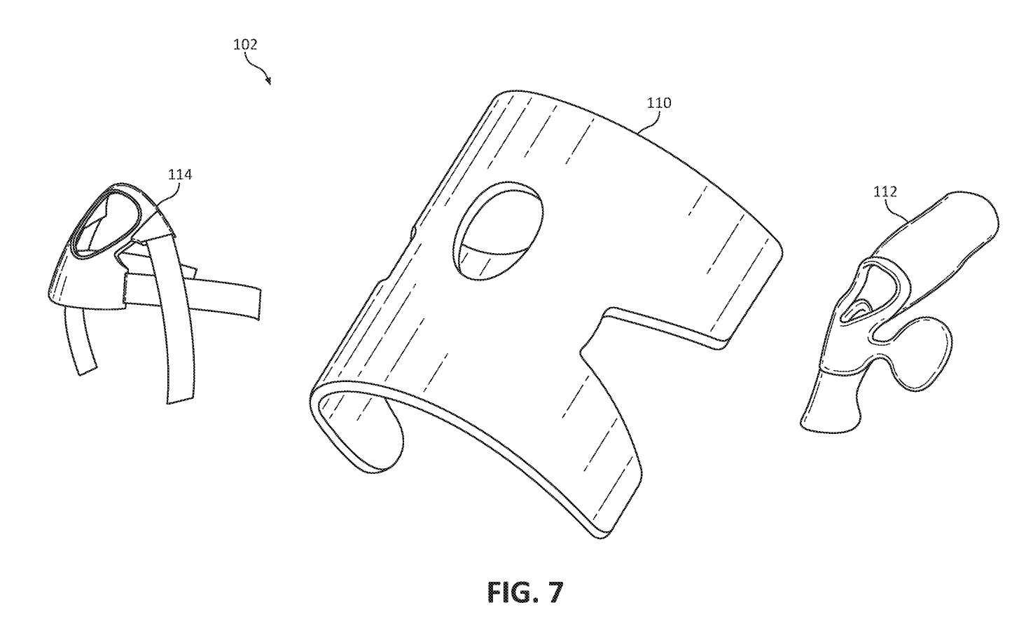

FIG. 7 illustrates a support assembly 102 incorporating an alternative sleeve 110 that may be used with the support assembly 102, according to some embodiments. The support assembly 102 includes a sleeve 110, an insert 112 and a strap 114. In general terms, the sleeve 110 is configured to maintain the insert 112; and the strap 114 assists with maintaining a position of the insert 112 and provides additional support as desired. The insert 112 is optionally molded and designed to help support various joint injuries and can be provided with a unique design and shape for a variety of injuries. The strap 114 holds the insert against a wearer's joint and provides adjustable tension to retain the insert 112 in a desired position.

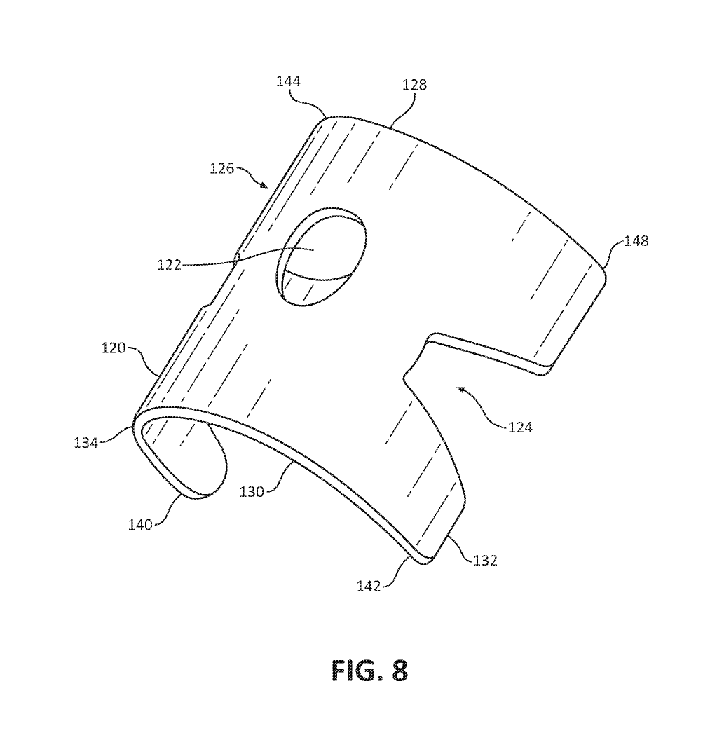

As shown in FIG. 8, in some embodiments, the sleeve 110 is configured as a wrap that is able to be wrapped about a limb of a wearer (not shown). The sleeve 110 includes a body 120. The body 120 defines an opening 122 corresponding to a kneecap, or patella of a wearer (not shown), for example. Alternatively, the opening 122 corresponds to an elbow or other anatomical feature of the wearer. The sleeve 110 defines a first face 124, a second face 126, a top 128, a bottom 130, a first side 132, and a second side 134. The sleeve 110 includes a plurality of arms 140, 142, 144, 148 extending from the body 120. In some embodiments, a sleeve 110 configured as a wrap may be worn by placing the first face 124 against a wearer's limb, for example an arm or leg, and wrapping the arms 140, 142, 144, 148 around the wearer's limb and securing the arms 140, 142, 144, 148 to one another or to the body 120 such that at least a portion of the limb of the wearer is received within the sleeve 110. For example, a sleeve 110 may be transitionable from a flat configuration to a generally tubular configuration by securing the arms 140, 142, 144, 148 to one another and/or the body 120 of the sleeve 110.

In some embodiments, the arms 140, 142, 144, 148 of the sleeve 110 may be joined together to form a sleeve 110 that fully encloses at least a portion of a wearer's limb. Optionally, the arms 140, 142, 144, 148 may be joined together in an adjustable variety of places depending on the wearer's desired configuration. For example, a wearer may join the arms 140, 142, 144, 148 at the top 128 and the bottom 130 of the sleeve 110, leaving a space in the middle. The arms 140, 142, 144, 148 are joined together using any suitable attachment device. In some embodiments, the arms 140, 142, 144, 148 are joined together with a clasp or fastener device. In another example, the arms 140, 142, 144, 148 are joined together using hook and loop fasteners sold under the trade name “VELCRO,” or other refastenable material.

In some embodiments, the sleeve 110 is made of any material that will allow the sleeve 110 to remain snug against a wearer's limb. For example, a sleeve 110 is made of elastic or stretchable material that may compress around a wearer's limb. In another example, a sleeve 110 is made of resilient material that does not stretch. The sleeve 110 is adjustable so a wearer may increase or decrease the compression to a joint as the wearer desires.

In some embodiments, the sleeve 110 is constructed to be worn on either a left or right limb. Optionally, the sleeve 110 may be constructed as a “universal sleeve” that can be worn on either a wearer's left or right limb. In some embodiments, the sleeve 110 is a universal size that may be worn by a number of wearers with varying dimensions. In some embodiments, the sleeve 110 is size adjustable such that a single sleeve 110 may be worn by a wearer of any height or weight. The sleeve 110 can be readjusted in size by manipulating where the arms 140, 142, 144, 148 join, thus one sized sleeve 110 can be used by wearers with varying limb widths.

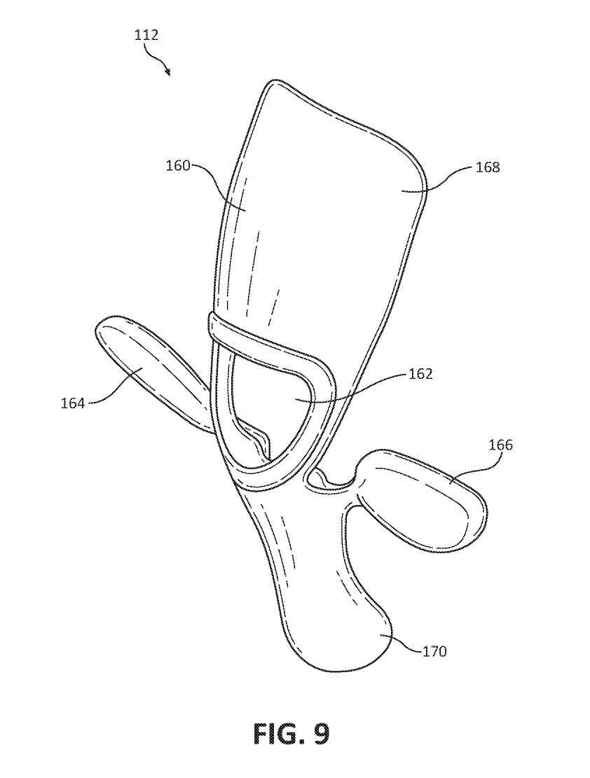

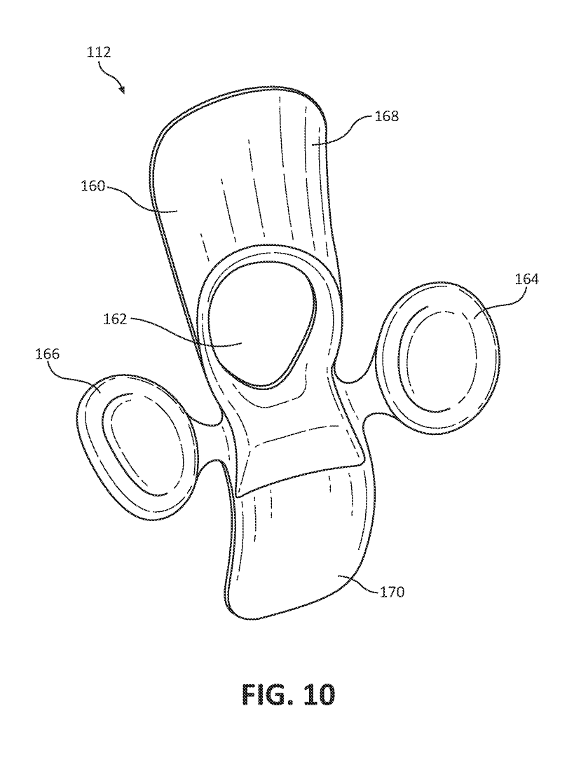

For illustrative purposes, FIGS. 9, 10 and 11 show an example insert 112 that is used with some embodiments of the support assembly 102 shown in FIG. 7. In some embodiments, an insert 112 is constructed to conform to the shape of the outer surface of a wearer's limb or joint, and provides support and/or pressure along certain areas of the limb or joint. In some embodiments, the insert 112 is constructed to support part of a limb or joint from one or multiple sides. In some embodiments, the insert 112 may completely encompass a wearer's limb or joint. In some embodiments, the insert 112 is constructed to cover the medial and lateral sides of a wearer's knee. In some embodiments, the insert 112 is constructed to be positioned on the medial and lateral sides of a wearer's knee beside the knee joint and the kneecap. In some embodiments, the insert 112 is constructed to be positioned superior and inferior to the wearer's knee or kneecap. In some embodiments, the insert 112 is constructed to be positioned on the anterior side of the leg, superior and inferior to the knee joint and the kneecap. The insert 112 shown in FIGS. 9, 10, and 11 incorporates similar elements as the previously described insert 12 and numbered elements shown in FIGS. 3, 4, and 5, correspond to the numbered elements in FIGS. 9, 10, and 11, now preceded with a “1.”

As shown in FIGS. 9, 10, and 11, in some embodiments, the insert 112 is constructed with features shaped to be positioned against various parts of a wearer's limb and joint. The insert 112 includes a main body 160 that provides support to a wearer's leg and knee. The main body 160 optionally defines an opening 162 that corresponds to the knee cap or patella of a wearer. In some embodiments, an opening 162 corresponds to the wearer's elbow, wrist, or ankle.

In some embodiments, the insert 112 includes support elements 164, 166, 168, 170 attached to the main body 160. The support elements 164, 166, 168, 170 are shaped to conform, for example, to the lateral, medial, superior, and inferior sides of a wearer's knee respectively. The support elements 164, 166, 168, 170 are suitably shaped and positioned to support the knee cap and knee joint.

In some embodiments, the support elements 164, 166, 168, 170 provide pressure to certain points along a limb or joint. For example, the support elements 164, 166, 168, 170 are configured to apply pressure from the lateral, medial, superior, or inferior sides of the knee joint, respectively, to increase support for the joint. In some embodiments, the insert 112 is constructed to prevent certain parts of the joint, such as the kneecap, from being pressed against the joint during physical activity. For example, the opening 162 allows parts of a wearer's joint to be positioned through the opening 162 for additionally fit, and have support elements 164, 166, 168, 170 positioned around or cradle suitable parts of a wearer's joint. As shown in FIGS. 9, 10, and 11, support elements 164, 166 that correspond to the lateral and medial sides of the wearer's knee are positioned in the plane of the main body 160. However, when worn, support elements 164, 166 are turned inward, so they are positioned perpendicular to the plane of the main body 160, and lay flat against the sides of the knee.

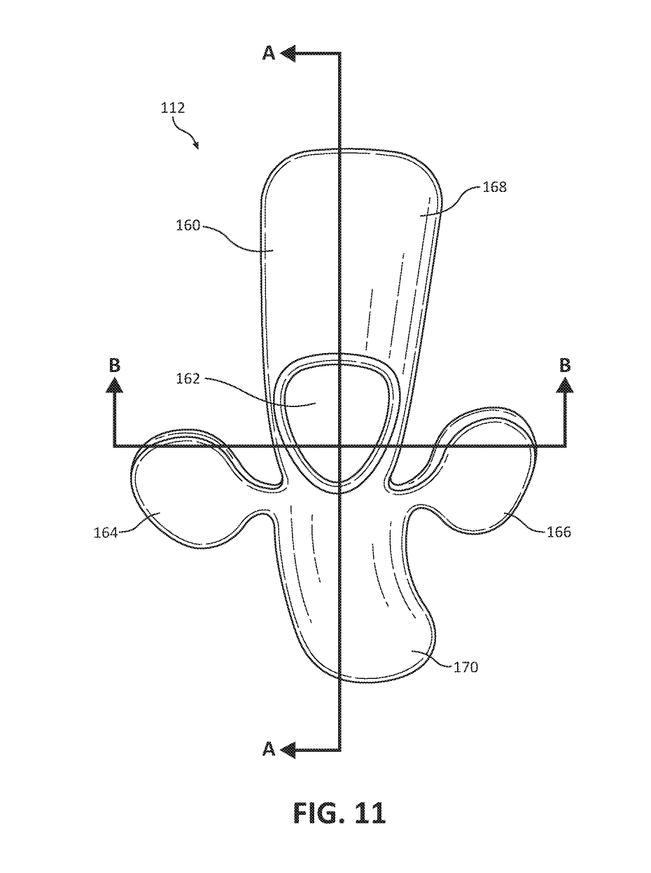

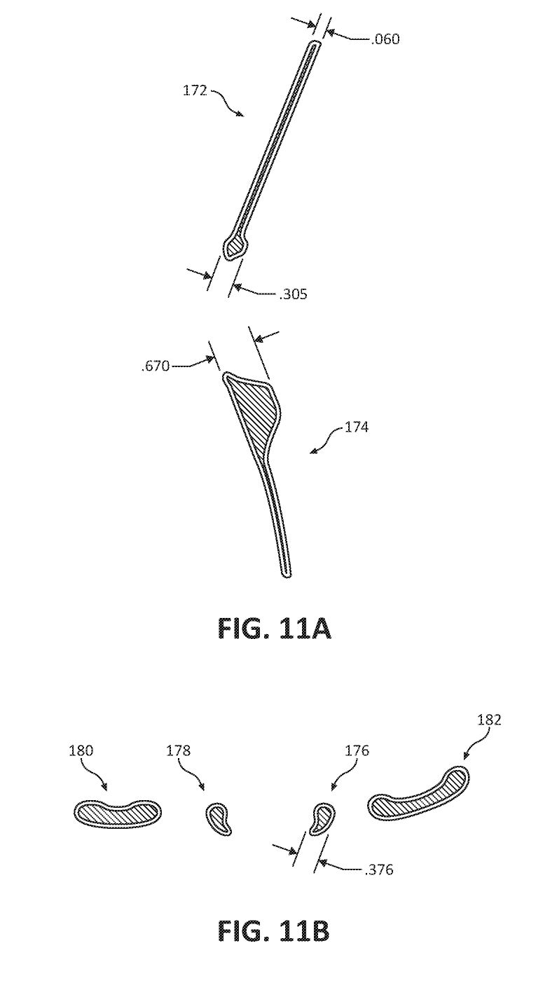

FIG. 11A is a cross-sectional view of the insert 112 in the plane of the lines marked by arrows A-A in FIG. 11. As shown in FIG. 11A, in some embodiments, the thickness of the insert 112 varies in specific locations to provide pressure to certain points along a wearer's knee cap, leg, and knee joint. For example, a portion of the insert 112 corresponding to the area superior 172 to the wearer's knee corresponds to the profile of the space superior to the wearer's kneecap between the kneecap and leg. Additionally, the portion of the insert 112 corresponding to the area inferior 174 to the wearer's knee corresponds to the profile of the space inferior to the wearer's kneecap between the kneecap and leg.

FIG. 11B is a cross-sectional view of the insert 112 in the plane of the lines marked by arrows B-B in FIG. 11. As shown in FIG. 11B, in some embodiments, the thickness of the insert 112 varies in specific locations to provide pressure to certain points along a wearer's knee cap, leg, and knee joint. For example, a portion of the insert 112 may be configured to correspond to the profile of the space medial 178 to the wearer's knee cap and leg. Additionally, a portion of the insert 112 may be configured to correspond to the profile of the space lateral 176 to the wearer's knee cap and leg. Support elements 164, 166 of the insert 112 (shown in FIG. 11) also have a thickness that provides support when the insert 112 is positioned against a wearer's knee. As shown in FIG. 11B, a cross-section view of the insert of FIG. 11 illustrates the shaped thickness of third and fourth support elements 164, 166 (shown in FIG. 11) that allow them to support a wearer's knee from the medial 180 and lateral 182 sides of the wearer's knee.

In some embodiments, the insert 112 is constructed to conform to the outer shape of a wearer's elbow, ankle, or wrist. In some embodiments, the insert 112 is constructed to fit around the wrist of a wearer and provide support to the wrist bones. In another example, the insert 112 is constructed to fit in the palm, around the thumb, or any other area of a wearer's hand. In another example, the insert 112 is constructed to support a wearer's ankle.

In some embodiments, the insert 112 is an article that has been custom molded to fit a particular wearer's joint. For example, a potential wearer's anatomy, such as a leg, arm, knee, elbow, or torso is scanned to determine the outer shape of the body part. Using data from the scan, a three dimensional model is created that corresponds to the outer surface of the scanned body part. The corresponding model is then used to design a custom molded insert 112 for the particular shape of a particular user's joint.

The three dimensional model may also be used to determine what shape an insert 112 must be in order to provide custom support to a particular area of a wearer's body part. For example, if a potential wearer has suffered an injury that requires greater support or pressure at a particular area, a scan of the wearer's anatomy provides the necessary location of pressure points. The three dimensional model is then used to design and mold an insert 112 that corresponds to provide additional support to these pressure points. Examples of some methods of forming pads suitable for use as inserts are provided in U.S. Patent Application No 2011/0056004, the entire contents of which are incorporated herein by reference.

In some embodiments, the insert 112 is held in place by joining the insert 112 to the sleeve 110. For example, the sleeve 110 may have pockets or compartments for holding an insert 112. In some embodiments, the sleeve 110 has compartments on the first face 124 of the sleeve 110 such that a user may place an insert 112 inside these compartments. In some embodiments, the insert 112 is attached to the sleeve 110 by a fastener such as a clasp or clip. In some embodiments, the sleeve 110 is constructed with straps or belts for holding an insert 112 in place. The pockets or compartments are constructed to hold the insert 112 in a particular positon against the wearer's limb or joint. In some embodiments, the sleeve 110 is constructed to hold the insert 112 in a fixed position relative to the sleeve 110. In some embodiments, the sleeve 110 is constructed to allow the insert 112 to be repositioned with respect to the sleeve 110. Optionally, the sleeve 110 is constructed to hold a variety of inserts 112, such that the same sized sleeve 110 may be used by a number of wearers.

In some embodiments, the sleeve 110 is constructed with compartments configured to hold an insert 112 in position against the wearer's limb or joint while the wearer is engaged in physical activity. For example, the compartments may be made from elastic or flexible material that allows the main body 120 of the sleeve 110 to move with the wearer's body yet maintains the insert 112 in place to provide support for the wearer's joint.

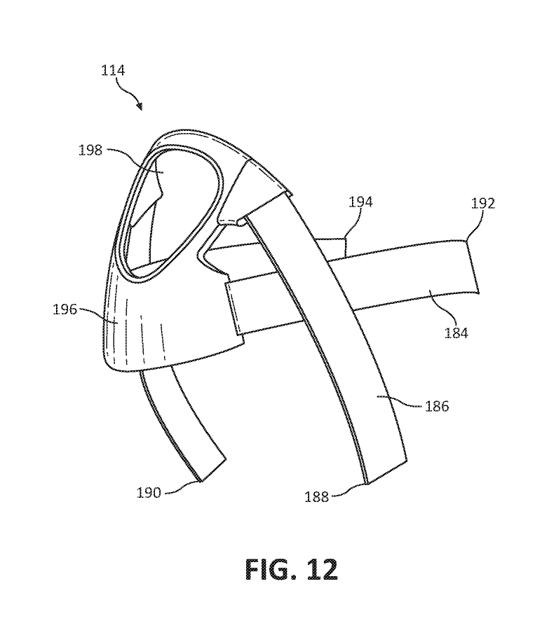

In some embodiments, a strap 114 is used in conjunction with the sleeve 110 to hold the insert 112 in position. FIG. 12 shows a perspective view of a strap 114. The strap 114 is used in conjunction with the sleeve 110 to apply adjustable tension, fit, and support to the wearer's limb or joint. The strap 114 is also used to hold the insert 112 in place against the wearer's limb or joint. In some embodiments, the strap 114 is constructed to apply adjustable tension such that the insert 112 is held snug against a wearer's limb. In some embodiments, the sleeve 110 is constructed with a strap 114 attached to the sleeve 110. In some embodiments, the strap 114 may be constructed as a separate component from the sleeve 110. The strap shown in FIG. 12 and described herein incorporates similar elements as the previously described strap 12 and similar elements as those shown in FIG. 6 correspond to numbered elements in FIG. 12 now preceded with a “1.”

In some embodiments, the strap 114 is attached to the sleeve 110 along the entire length of the strap 114. In some embodiments, the strap 114 is attached to the sleeve 110 only in certain locations. For example, the strap 114 has portions that are not attached to the sleeve 110, thus allowing the strap 114 to move independent of the sleeve 110 at the unattached locations. In some embodiments, the strap 114 attaches to the sleeve 110 in one or more places and applies tension between the attached locations.

The strap 114 may be attached to the sleeve 110 by any suitable attachment method such as sewing, or using clips, clasps, hook and loop fasteners, sold under the trade name “VELCRO,” or buttons, for example. In some embodiments, the strap 114 is configured to allow a wearer to partially or completely remove the strap 114 from the sleeve 110, for example to change the position where the strap 114 connects to the sleeve 110.

In some embodiments, the strap 114 is a band positioned around the insert 112 and the sleeve 110. The band thus applies compression to the insert 112 in a direction normal to the axial direction of a wearer's limb and holds the insert 112 against the wearer's limb. In some embodiments, the strap 114 is constructed such that it wraps completely around the sleeve 110 and a wearer's limb or joint. Alternatively, the strap 114 is constructed to wrap partly around the wearer's limb or joint.

As illustrated in FIG. 12, in some embodiments, the strap 114 may include multiple bands. For example, the strap 114 is constructed with a first band 184 that travels under or inferior to a knee cap, and a second band 186 that travels over or superior to a knee cap. When using multiple bands to form a strap system, a variety of optional configurations are possible for positioning the first and second band 184, 186 in relation to the joint and insert 112. For example, the first and second band 184, 186 may cross over each other forming an X in the front of a knee cap, or can cross on the sides of the knee. In another example, the first and second band 184, 186 may be constructed to each have first ends 188, 192 attach at the medial or posterior side of a wearer's knee, traverse the anterior portion of the knee and each have second ends 190, 194 attach to the posterior or lateral side of the knee.

In a strap 114 with multiple bands 184, 186, the bands 184, 186 work in conjunction to keep the insert 112 in a suitable position. The strap 114 holds the insert 112 in place by preventing movement in a first axial direction along the wearer's limb with the first band 184 and a second axial direction along the wearer's limb with the second band 186.

In some embodiments, the strap 114 is configured to correspond to the outer shape of the insert 112 and provide a complimentary fit to the insert 112. The strap 114 is optionally configured to be positioned within shaped depressions in the outer surface of the insert 112 to further assist the strap 114 in preventing lateral or torsional movement of the insert 112 in relation to the wearer's joint.

In some embodiments, a retaining portion 196 is constructed between the first band 184 and second band 186 and connects the first band 184 and the second band 186. The retaining portion 196 is configured to correspond to the outer shape of the insert 112 such that the strap 114 prevents lateral or torsional movement of the insert 112 in relation to the wearer's limb. In some embodiments, the retaining portion 196 defines an opening 198 to allow parts of a wearer's joint to be positioned inside the opening. The opening 198 is shaped to support a kneecap or patella, or an ankle, elbow, or wrist. The opening 198 is configured to correspond to the outer shape of the insert 112 and retain it in position against the wearer's limb or joint.

In some embodiments, the strap 114 is configured to allow a wearer to adjust the tension applied by the strap 114 to the wearer's limb or joint. For example, the strap 114 allows a wearer to adjust the length of the strap 114 using a clasp or clip. Alternatively, the strap 114 is repositionable with hook and loop fasteners, to allow a wearer to reposition where ends 188, 190, 192, 194 of the strap 114 attach to the sleeve 110. Using repositionable attachments allows a wearer to reposition the strap 114 at various locations on the sleeve 110 and thus allows the wearer to adjust the position of the strap 114 in relation to the wearer's limb. The wearer can also adjust the angle of the strap 114 in relation to the wearer's limb or joint and adjust the strap tension and the pressure points on the insert 112.

When worn, the support assembly 102 includes the sleeve 110, insert 112, and strap 114 coupled together. A suitable insert 112 is chosen for a user's particular needs. The insert 112 is a pad molded or designed to help support different joint injuries with a unique design for each particular problem. The insert 112 is constructed to be used with a variety of sleeves and may be provided separately from the support assembly 102. The insert 112 is placed into the sleeve 110 by the wearer and held in place using connectors, pockets, clasps, or clips. The sleeve 110 containing the insert 112 is placed onto a wearer's limb and fastened around a wearer's joint by attaching the arms 140, 142, 144, 148 to one another or to the sleeve body 120. The strap 114 is positioned around the sleeve 110 and insert 112 and connected to the sleeve 110 to supply adjustable tension, fit, and support to the sleeve 110 and insert 112, and holds the insert 112 in place against the wearer's joint.

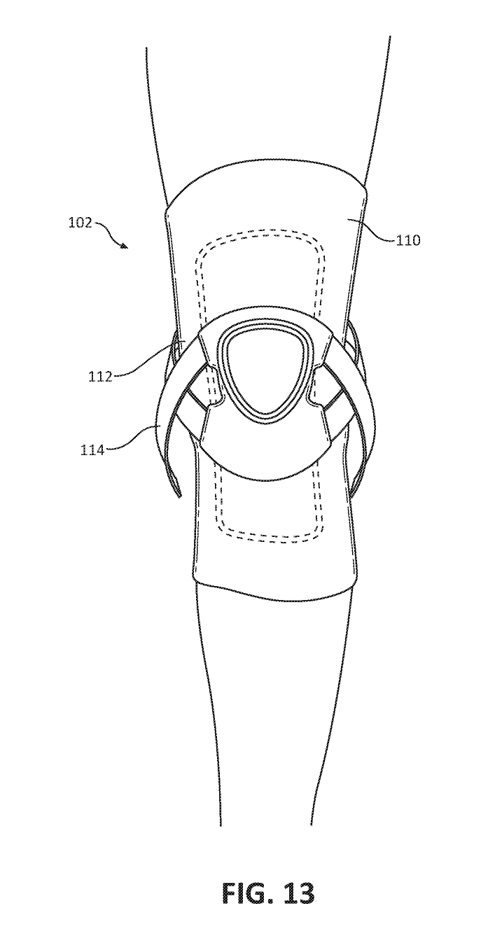

FIG. 13 is a front view of the support assembly 102 as assembled on a wearer's limb. As shown in FIG. 13, the sleeve 110 is placed onto a wearer's limb to provide support to the wearer's limb. The sleeve 110 also hold the insert 112 in place against the wearer's joint to support and stabilize the joint. The strap 114 is placed over the sleeve 110 and the insert 112 and provides additional support for keeping the insert 112 in place against the wearer's limb. In this configuration, the various components of the support assembly 102 combine to provide a support to a wearer, and may provide tailored support to areas of a wearer's limb that may have been injured or require particular support. The support assembly 102 is also flexible or pliable to allow a wearer to engage in physical activity without the support assembly 102 restricting the wearer's movement.

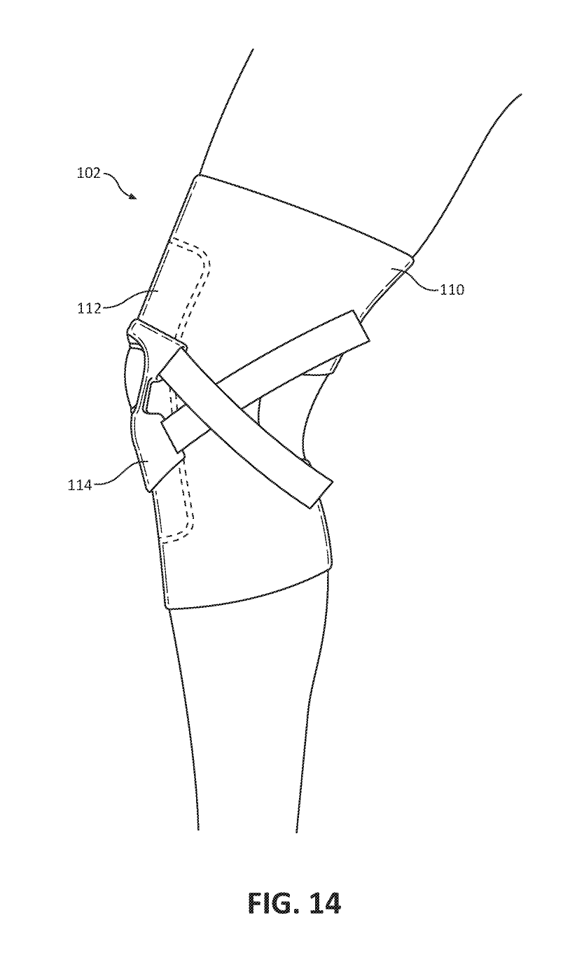

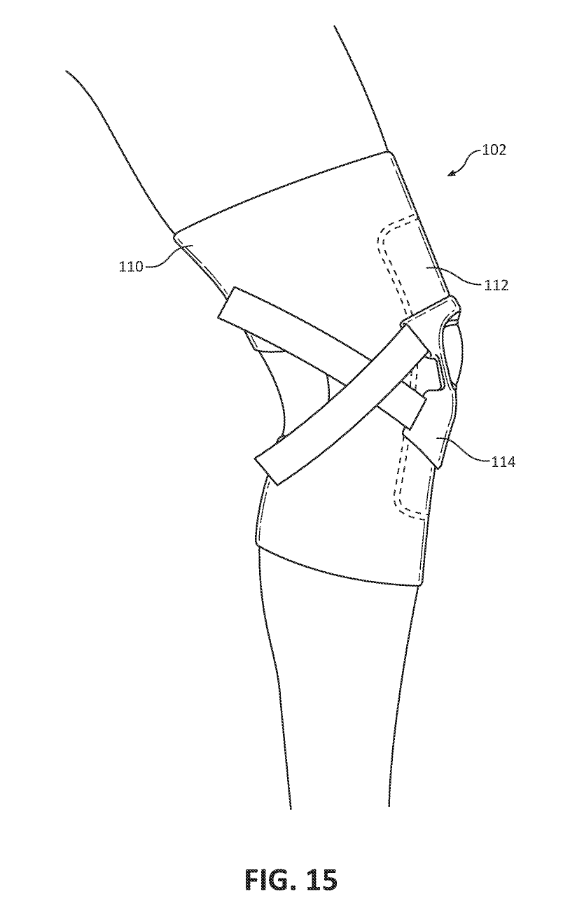

FIG. 14 is a first side view of the support assembly 102 as assembled on a wearer's limb. FIG. 15 is a second side view of the support assembly 102 as assembled on a wearer's limb. As shown in FIGS. 14 and 15, when worn, the sleeve 110 sits snugly against a wearer's limb and reduces movement of the insert 112 in relation to the wearer. The strap 114 also provides additional support to the sleeve 110 and assists in holding the insert 112 in place against a wearer's limb.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the above described features.

The following is claimed:

1. A support assembly, comprising:
a sleeve configured to surround a portion of a limb of a wearer; the sleeve having a main body defining a first opening, a second opening, an inner surface, and an outer surface, wherein the first and second openings are configured to receive a limb of a wearer;
a support insert configured to engage the sleeve and be held in position against a limb of a wearer by the sleeve; and
a strap configured to be positioned around the support insert and sleeve and retain the support insert against the portion of a limb of a wearer.

2. The support assembly of claim 1, wherein the sleeve is configured to be interchangeably worn on a wearer's left limb or right limb.

3. The support assembly of claim 1, wherein the support insert is molded from a flexible foam material.

4. The support assembly of claim 1, wherein the support insert is constructed to form a complimentary fit to an outer surface of a wearer's joint.

5. The support assembly of claim 1, wherein the support insert is constructed to form a complimentary fit to an outer surface of a wearer's knee.

6. The support assembly of claim 1, wherein the support insert is constructed to conform to the outer surface of a wearer's knee such that the support insert provides weight bearing support.

7. The support assembly of claim 1, wherein the strap is configured to provide adjustable tension to hold the support insert against a wearer's limb.

8. The support assembly of claim 1, wherein the strap is constructed to attach to the outer surface of the sleeve.

9. An athletic brace, comprising:
a sleeve configured to surround at least a portion of a wearer's limb, and configured to provide stabilizing support to the portion of a wearer's limb;
a support pad molded to conform to an outer shape of the portion of a wearer's limb, the support pad configured to be held in place along the portion of a wearer's limb by the sleeve; and
a strap assembly configured to attach to the sleeve and reinforce the support pad against the portion of a wearer's limb.

10. The athletic brace of claim 9, wherein the sleeve is configured to be interchangeably worn on a wearer's left or right limb.

11. The athletic brace of claim 9, wherein the support pad is molded from a flexible foam material.

12. The athletic brace of claim 9, wherein the support pad is constructed to conform to an outer surface of a wearer's knee.

13. The athletic brace of claim 9, wherein the support pad is constructed to conform to an outer surface of a wearer's knee such that the protective pad provides weight bearing support to the knee.

14. The athletic brace of claim 9, wherein the strap assembly is configured to provide adjustable tension to the support pad against a wearer's limb.

15. A support system for a limb of a wearer, comprising:
a wrap having a first and second opening and an inward facing surface and an outward facing surface;
a cushioned insert;
a strap system configured to engage the outward facing surface of the resilient wrap; wherein
the support system is constructed such that the wrap is configured to surround a limb of a wearer and provide a compressive force to the limb of a wearer,
the cushioned insert is held against the limb of a wearer by the compressive force provided by the wrap, and
the strap system maintains the cushioned insert in position against the limb of a wearer.

16. The support system of claim 15, wherein the wrap is configured to be interchangeably worn on a wearer's left or right limb.

17. The support system of claim 15, wherein the cushioned insert is molded from a flexible foam material.

18. The support system of claim 15, wherein the cushioned insert is constructed to conform to an outer surface of a wearer's knee.

19. The support system of claim 15, wherein the cushioned insert is constructed to conform to an outer surface of a wearer's knee and provide weight bearing support to the knee.

20. The support system of claim 15, wherein the strap system is configured to provide adjustable tension to the cushioned insert against the limb of a wearer.
